(19) United States
(12) Patent Application Publication
(10) Pub. No.: US 2024/0161908 A1
(43) Pub. Date: May 16, 2024

(54) METHOD FOR PROVIDING AT LEAST ONE FIRST METADATA ATTRIBUTE COMPRISED BY MEDICAL IMAGE DATA

(71) Applicant: Siemens Healthcare GmbH, Erlangen (DE)

(72) Inventor: Ananda HEGDE, Erlangen (DE)

(73) Assignee: Siemens Healthcare GmbH, Erlangen (DE)

(21) Appl. No.: 18/499,529

(22) Filed: Nov. 1, 2023

(30) Foreign Application Priority Data
Nov. 3, 2022 (DE) 10 2022 211 617.8

(51) Int. Cl. G16H 30/40; G06F 40/40; G16H 30/20

(52) U.S. Cl. CPC G16H 30/40; G06F 40/40; G16H 30/20

(57) ABSTRACT

One or more example embodiments of the present invention describes a computer-implemented method for providing at least one first metadata attribute associated with medical image data. The method comprises receiving the medical image data and the at least one first metadata attribute, the at least one first metadata attribute including an attribute tag and a provisional attribute value; applying a first trained function to the medical image data to determine a standardized attribute value; determining a final attribute value based on the provisional attribute value and the standardized attribute value; and providing the at least one first metadata attribute, the at least one first metadata attribute including the attribute tag and the final attribute value.

### CROSS-REFERENCE TO RELATED APPLICATION(S)

The present application claims priority under 35 U.S.C. § 119 to German Patent Application No. 10 2022 211 617.8, filed Nov. 3, 2022, the entire contents of which are incorporated herein by reference.

### FIELD

One or more example embodiments of the present invention describes a method and a system for providing at least one first metadata attribute comprised by medical image data.

### RELATED ART

Medical images of an examination object can be acquired during an examination or during a diagnostic process. The medical image is often shared or stored as medical image data. The medical image data are based on at least one medical image and/or the medical image data comprise the medical image. The examination object can at least comprise a part respectively an organ of a human being, an animal and/or an object. It is known that at least one metadata attribute is associated with a medical image. The medical image data often comprise at least one metadata attribute, wherein the metadata attribute is based and/or associated with a medical image, wherein the medical image is comprised by the medical image data and/or the medical image data are based on it.

The metadata attribute is designed for describing for example to which examination, which diagnostic process, which examination object and/or which part of the examination objects the associated medical image is related to. Alternatively, or additionally, the metadata attribute can precise at least one examination parameter which has been used for acquiring the medical image. A metadata attribute usually comprises an attribute tag and an attribute value. The attribute tag describes what the metadata attribute refers to. The attribute value provides the concrete value of the metadata attribute. The attribute value is usually determined automatically and/or manually. For example, the metadata attribute can be filled in manually by a medical doctor or a medical assistant or a technologist during the imaging process. Filling in the metadata attribute means that the attribute value is filled in by a string or a digit (e.g. an integer, a float etc.). Alternatively or additionally, the metadata attribute can be determined automatically. For example, the metadata attribute can be filled in based on a protocol which has been used for acquiring the associated medical image.

In healthcare environment at the end of every scan or exam an electronic document is normally produced. The electronic document is often based on the DICOM standard. Electronic health record (EHR) systems are a rich source of data accumulated through routine clinical care and secondary use of EHR data for analytics, research, quality and safety measurement. The Health Information Technology for Economic and Clinical Health (HITECH) Act and the meaningful use incentive program facilitated widespread EHR adoption. Consequently, multi-site data aggregation and centralization are feasible and increasingly common. These aggregate data sources are important for research, quality, public health, and commercial applications.

Operator often fill important DICOM tag values in their own regional/local languages based on their knowledge. Scanner operators or admins may not be educated or aware of standards which should be fed to the electronic record. This would lead to a problem for the Radiologist from other location or region of the globe and DICOM study or electronic document cannot be used commonly across the globe. Radiologist may not be able to assist or analyze the problem and derive the solution.

In some cases, the metadata attribute can be filled in incorrectly and/or incomplete. For example, a protocol for imaging a hand is used for imaging a foot. The attribute value of the metadata attribute with the attribute tag ‘Body part examined’ is automatically filled by the string ‘foot’. As an alternative example the medical doctor or the medical assistant forgot to fill in a metadata attribute. Alternatively or additionally, the metadata attribute might be filled in with an abbreviation or an individual expression instead of a standardized label when it is filled in manually.

Because of this it might be impossible to compare different medical images in a database based on the metadata attributes. For example, querying for all medical images which are associated to a metadata attribute with an attribute tag ‘Body part examined’ and an attribute value ‘foot’ might result according to the example from above to a plurality of medical images wherein at least one comprises an image of a hand. Furthermore, if the attribute value is filled manually, it might not be possible to query for a specific group of medical images based on the attribute values, because the attribute values are not standardized.

Furthermore, an application which is applied on the medical image might need an information based on the correctly filled attribute value of a metadata attribute. For example, an application which provides a diagnosis or makes automated measurements based on the medical image might need an information about the examination object. This information should be provided by the corresponding correct and/or standardized attribute value.

Mapping medical test names into a standardized vocabulary is therefore a prerequisite for sharing test-related data between health care entities. One major barrier in this process is the inability to describe tests in sufficient detail to assign the appropriate name. Hence some standards are critical for interoperability (like RPID) and integrating data into common data models but are inconsistently used. Without consistent mapping to standards, clinical data cannot be harmonized, shared, or interpreted in a meaningful context.

### SUMMARY

Empty, incorrect and/or not standardized attribute values can cause problems concerning the further processing of the medical image data based on the at least one metadata attribute. Hence, the metadata attributes and their corresponding attribute values should be unified. One or more example embodiments of the present invention is especially based on an automated machine learning pipeline that leverages noisy labels to map attribute values and/or attribute tags to standard names or codes.

One or more example embodiments of the present invention provides an automatically translation of local medical terms to the common standard, so that the electronic document, especially the medical image data, could be shared across any health care entities or radiologists without any decoding of details.

One or more example embodiments of the present invention provides a method unifying a metadata attribute, especially an attribute tag and/or an attribute value, associated to a medical image and/or image data.

A method, a unifying system, a computer program product and a computer-readable storage medium according to the independent claims for unifying a metadata attribute are disclosed. Advantageous features and further developments are listed in the dependent claims and in the following specification.

### BRIEF DESCRIPTION OF THE DRAWINGS

Objects and features of example embodiments of the present invention will become apparent from the following detailed descriptions considered in conjunction with the accompanying drawings. It is to be understood, however, that the drawings are designed solely for the purposes of illustration and not as a definition of the limits of the invention.

FIG. 1 displays a schematic flow chart of a first embodiment of the method for providing at least one first metadata attribute associated with medical image data,

FIG. 2 displays a schematic flow chart of a second embodiment of the method for providing at least one first metadata attribute associated with medical image data,

FIG. 3 displays a schematic flow chart of a third embodiment of the method for providing at least one first metadata attribute associated with medical image data,

FIG. 4 displays a schematic flow chart of a fourth embodiment of the method for providing at least one first metadata attribute associated with medical image data,

FIG. 5 displays a unifying system for providing at least one first metadata attribute associated with medical image data according to one or more example embodiments,

FIG. 6 displays a further example of a unifying system for providing at least one first metadata attribute associated with medical image data according to one or more example embodiments,

FIG. 7 displays a further example of a unifying system and method flowchart for providing at least one first metadata attribute associated with medical image data according to one or more example embodiments, and

FIG. 8 displays an example of a natural language processing algorithm as part of a first trained function according to one or more example embodiments.

### DETAILED DESCRIPTION

In the following the solution according to one or more example embodiments of the present invention is described with respect to the claimed unifying systems as well as with respect to the claimed methods. Features, advantages or alternative embodiments herein can be assigned to the other claimed objects and vice versa. In other words, claims for the unifying systems can be improved with features described or claimed in the context of the methods. In this case, the functional features of the method are embodied by objective units of the unifying system. Independent of the grammatical term usage, individuals with male, female or other gender identities are included within the term.

In a first aspect, one or more example embodiments of the present invention relates to a computer-implemented method for providing at least one first metadata attribute comprised by medical image data and/or associated with medical image data. The medical image data are configured as a medical image and/or comprise at least one medical image. The method comprises a step of receiving the medical image data and the at least one first metadata attribute, wherein the at least one first metadata attribute comprises an attribute tag and a provisional attribute value. In a further step, the method comprises applying a first trained function to the medical image data so as to determine a standardized attribute value. In a further step, the method comprises determining a final attribute value based on the provisional attribute value and the standardized attribute value. In a further step, the method comprises providing the at least one first metadata attribute, wherein the at least one first metadata attribute comprises the attribute tag and the final attribute value.

The medical image data and/or the medical image can in particular be acquired by a medical imaging system. The medical imaging system can for example be one out of an X-ray system, a Computed-Tomography (CT) system, a Magnetic-Resonance-Imaging (MRI) system, an angiography system, a C-arm system, an ultrasonic system, a Positron-Emission-Tomography (PET) system, a Single-Photon-Emission-Computed-Tomography (SPECT) system. The medical image can in particular be a pixel image or a voxel image. With other words, the medical image can comprise a pixel matrix or a voxel matrix, wherein the pixel matrix respectively the voxel matrix comprises a plurality of pixels respectively voxels. Hence, the medical image of the medical image data can be a two-dimensional or a three-dimensional medical image. Alternatively, the medical image of the medical image data can be a four-dimensional medical image. A four-dimensional medical image can comprise a time-series of three-dimensional medical images. Such a four-dimensional medical image can be for example a Cine-Magnetic-Resonance-Image. Alternatively, a four-dimensional medical image can for example comprise a cardiac gated Computed-Tomography scan. Especially, the phrase medical image can be used for medical image data, wherein medical image means data comprising the medical image and the first metadata attribute.

The medical image and/or the medical image data can depict an examination object. The examination object can be at least a part or an organ of a human being or an animal or a subject.

The medical image data and/or the medical image is associated to at least one first metadata attribute. In particular, the medical image data and/or medical image can be associated to a plurality of metadata attributes, wherein the at least one first metadata attribute is one of the plurality of metadata attributes. The at least one first metadata attribute describes for example what is depicted by the medical image, by which medical imaging system the medical image is acquired and/or which parameters are used for acquiring the medical image. In particular, the at least one metadata attribute can comprise an information which part of the examination object is depicted in the medical image and/or in which orientation the part of the examination object is depicted and/or about the laterality of the part of the examination object and/or in which view the part of the examination object is imaged.

The at least one first metadata attribute can be comprised by a Digital Imaging and Communications in Medicine (DICOM) header which is associated to the medical image and/or medical image data. With other words, the at least one first metadata attribute can be a DICOM attribute which is comprised by a DICOM header of the medical image data and/or medical image. Alternatively, the at least one first metadata attribute can be comprised by a Neuroimaging Informatics Technology Initiative (NIfTI) header. Alternatively, the at least one first metadata attribute can be comprised by a header of the medical image, wherein the medical image can be of any other data format. The data format of the medical image can for example be Analyze or Medical Imaging Network Common Data Format (MINC).

The at least one first metadata attribute comprises an attribute tag and a provisional attribute value. The attribute tag specifies which information is comprised by the at least one first metadata attribute. For example, the attribute tag can be one out of the following: ‘Body part examined’, ‘Anatomic Region Sequence’, ‘Patient Orientation’, View Position’, ‘Image Laterality’, ‘Frame Laterality’, ‘Measurement Laterality’ etc. Typically, the attribute tag cannot be changed. The provisional attribute value provides the value corresponding to the attribute tag. In particular, the provisional attribute value can be empty. The provisional attribute value can comprise a string value and/or an integer value and/or a float value and/or a free-text value. In particular, the attribute value comprises an information about the “topic” which is provided by the corresponding attribute tag.

In the following, the expression “filling in the at least one metadata attribute” means that an attribute value which is comprised by the at least one first metadata attribute is filled in. Hence, “filling in the at least one first metadata attribute” and “filling in the attribute value” are used synonymously. The provisional attribute value can be filled in manually or automatically. In particular, the provisional attribute value can be filled in manually by a medical doctor and/or a medical assistant. Alternatively or additionally, the provisional attribute value can be filled in automatically by the medical imaging system. For example, the medical imaging system can fill in the provisional attribute value based on an imaging protocol.

In the step of receiving the medical image data and the at least one first metadata attribute, the medical image data and the at least one first metadata attribute is received by an interface. The medical image data and the at least one metadata attribute can be provided for example by a Picture Archiving and Communication System (PACS) or by a Radiology Imaging System (RIS) or by a Hospital Information System (HIS) etc.

In the step of applying the first trained function to the medical image data, the first trained function is applied to the medical image data, in order to determine the standardized attribute value. In particular, the standardized attribute value corresponding to the attribute tag can be determined based on the medical image data, further metadata attributes of the medical image data and/or the medical image. For example, by applying the first trained function it can be determined a standardized name, phrase or code which is common and/or international readable to describe the provisional attribute tag and/or value, e.g. to describe the procedure for acquiring the medical image, which part of the examination object is depicted by the medical image and/or it can be determined whether for example a right or a left hand is depicted by the medical image etc. For example, the user has filled in as provisional attribute tag “ [symbol] [symbol] [symbol] ” wherein applying the first trained function determines as standardized attribute value “Abdominal X-ray” or “RID33687”, wherein RID33687 is the RadLex-ID for the abdominal region. RadLex is a comprehensive set of radiology terms for use in radiology reporting, decision support, data mining, data registries, education and research.

In general, the first trained function mimics cognitive functions that humans associate with other human minds. In particular, by training based on training data the first trained function is able to adapt to new circumstances and to detect and extrapolate patterns.

In general, parameters of the first trained function can be adapted via training. In particular, supervised training, semi-supervised training, unsupervised training, reinforcement learning and/or active learning can be used. Furthermore, representation learning (an alternative term is “feature learning”) can be used. In particular, the parameters of the first trained function can be adapted iteratively by several steps of training.

In particular, the first trained function can comprise a neural network, a support vector machine, a decision tree and/or a Bayesian network, and/or the trained function can be based on k-means clustering, Q-learning, genetic algorithms and/or association rules. In particular, a neural network can be a deep neural network, a convolutional neural network, or a convolutional deep neural network. Furthermore, a neural network can be an adversarial network, a deep adversarial network and/or a generative adversarial network.

In particular, the first trained function can comprise a deep learning network (e.g. deep belief network, residual neural network, dense neural network, autoencoder, capsule network, generative adversarial network, Siamese network, convolutional neural network, image transformer network) respectively it can be based on a deep learning technique (e.g. deep reinforcement learning, landmark detection). Alternatively or additionally, the first trained function can be based on a machine learning technique (e.g. support vector machine, Bayesian model, decision tree, k-means clustering). Alternatively or additionally, the first trained function can be based on a traditional image processing technique (e.g. template matching, content based image retrieval, similarity search morphological processing), a text processing technique, an audio processing technique and/or an text processing technique.

The standardized attribute value can be determined based on an available ontology or a standardized dictionary. The available ontology can for example be a standard ontology like RadLex or Systematized Nomenclature of Medicine Clinical Terms (SNOMED-CT). The provisional attribute value, especially in a non-standardized ontology, can be based on an user defined dictionary which comprises abbreviations and/or acronyms and/or expressions typically used by the medical doctor, within a hospital and/or within a country, where the medical image is acquired.

In the step of determining the final attribute value, the final attribute value is determined based on the provisional attribute value and the standardized attribute value. In particular the final attribute value can be determined by a comparison of the provisional attribute value and the standardized attribute value. In particular, determining the final attribute value can be performed automatically. Alternatively, determining the final attribute value can be performed manually by the medical doctor and/or the medical assistant. For example, if the provisional attribute value is the same as the standardized value, the final attribute value is set as the provisional attribute value or the standardized value and/or if the provisional attribute value is in the standard langue and/or format and a synonym to the standardized value, the final attribute value is set as the provisional and/or standardized attribute value. In particular, if the provisional attribute value is not in standard format or language, the standardized attribute value is set as the final attribute value.

In the step of providing the at least one first metadata attribute, the at least one first metadata attribute is provided by the interface. In particular, the at least one first metadata attribute is provided in association with the medical image and/or medical image data. The at least one first metadata attribute comprises the attribute tag and the final attribute value. The at least one first metadata attribute can for example be used to query a plurality of medical images all comprising a metadata attribute comprising the same attribute value. Alternatively or additionally, the at least one first metadata attribute can be used together with the medical image for further diagnosis of the examination object. Alternatively or additionally, the at least one first metadata attribute can be used by an application which should be applied to the medical image. The application can for example be configured for image processing of the medical image. It might be necessary for applying the application to know the correct at least one first metadata attribute.

The inventors recognized that the attribute value of at least one metadata attribute associated to medical image data can be unified by applying the first trained function to the medical image data. In particular, the at least one first metadata attribute can be unified based on the associated medical image data, especially based on the provisional attribute tag. The first trained function is for example configured to translate and/or to map non-standardized attribute tags and/or non-standardized provisional attribute values into standardized ones, e.g., into a standardized language or according to a standardized dictionary. Especially, the first trained function is configured to determine the standardized attribute value based on analyzing the information comprised by the provisional attribute value, the attribute tag of the assigned metadata attribute and/or by analyzing the other metadata attributes of the medical image data. In particular unifying means in this context correcting or filling in the final attribute value of the at least one first metadata attribute value.

According to a further aspect of one or more example embodiments of the present invention the method comprises a step of checking whether the provisional attribute value is empty, wherein the step of determining the final attribute value is based on the result of the check. Alternatively and/or additionally the method comprises the step of checking whether the provisional attribute value is standardized, e.g. comprised by and/or according to the standardized ontology, dictionary or language, wherein the step of determining the final attribute value is based on the result of the check.

With other words, before determining the final attribute value based on the provisional attribute value and the standardized attribute value, it is checked whether the provisional attribute value is empty and/or whether the attribute value is standardized or not. Determining the final attribute value is based on this comparison. With other words the step of determining the final attribute value is adapted to the result of the check.

If the provisional attribute value is not empty, it is necessary to decide whether the provisional attribute value or the standardized attribute value or a combination of both should be set as the final attribute value in the step of determining the final attribute value. If it is empty, the final attribute value can be set to be the standardized attribute value. If the provisional attribute value is not standardized, the standardized attribute value is determined and/or the standardized attribute value is set as the final attribute value.

The inventors recognized that the step of determining the final attribute value can be dependent of the provisional attribute value. The inventors recognized that the final attribute value should be determined in dependence whether the provisional attribute value is empty or not and/or whether the provisional attribute value is already standardized or not.

According to a further aspect of one or more example embodiments of the present invention, if the provisional attribute value is empty and/or if the provisional attribute value is not standardized, the step of determining the final attribute value comprises a step of filling the final attribute value with the standardized attribute value.

With other words, if the provisional attribute value is empty or not standardized, the final attribute value is chosen to be the standardized attribute value. With other words, the final attribute value can be set to be the standardized attribute value if the provisional attribute value is empty or non-standardized.

The inventors recognized that the final attribute value can be filled with the standardized attribute value if the provisional attribute value is empty or non-standardized. The inventors recognized that like this, it is possible to fill the attribute value of a metadata attribute, which is not filled in automatically or manually during the imaging process and/or not filled standardized, automatically based on the medical image data. With other words, the inventors recognized that like this an autofilling based on the medical image data can be performed. The inventors recognized that this helps to avoid empty and/or non-standardized attribute values. Furthermore, the inventors recognized that the effort of the medical doctor or the medical assistant and/or a potential human error can be reduced by autofilling of the final attribute value according to one or more example embodiments of the present invention.

According to a further aspect of one or more example embodiments of the present invention, if the provisional attribute value is not empty, the step of checking if the provisional attribute value is standardized or not is carried out and/or applied. Particularly, if the provisional attribute value is not empty and non-standardized, the standardized attribute value is determined and set as the final attribute value. In particular, if the provisional attribute value is not empty but is standardized, the provisional attribute value is set as the final attribute value.

The inventors recognized that the provisional attribute value can be automatically corrected by choosing the standardized attribute value as final attribute value. The inventors recognized that this can be done automatically based on the method according to one or more example embodiments of the present invention.

According to a further aspect of one or more example embodiments of the present invention a plurality of metadata attributes is associated to the medical image data. Therein each metadata attribute of the plurality of metadata attributes comprises an attribute tag and an attribute value. Therein the first metadata attribute is one metadata attribute of the plurality of metadata attributes. Therein, the method further comprises the step of determining the at least one first metadata attribute out of the plurality of metadata attributes based on an application configured to process the medical image and/or on the attribute tags.

In particular, the plurality of metadata attributes describes respectively specifies the medical image. With other words a metadata attribute can describe a property of the medical image. Alternatively or additionally, a metadata attribute can describe a property the acquisition process of the medical image. In particular, each metadata attribute can describe another property of the medical image. Preferably, a metadata attribute describes the protocol, study description, series description, program, parameters and/or method of acquiring the medical image and/or medical image data. E.g., a metadata attribute describes the process of acquisition of the medical image data as “MR Brain without Contrast agent”. In particular, each metadata attribute of the plurality of metadata attributes can be designed like the at least one first metadata attribute. Wherein each metadata attribute relates to another context concerning the medical image.

In the step of receiving the at least one first metadata attributes all metadata attributes of the plurality of metadata attributes can be received. The at least one first metadata attribute can be determined out of the plurality of metadata attributes in the step of determining the at least one first metadata attribute.

The at least one metadata attribute can in particular be determined based on an application configured to process the medical image of the medical image data. In particular, the application can be configured to perform some image processing based on the medical image. The application can for example be configured to determine a diagnosis based on the medical image. The application can need the at least one first metadata attribute as input value. For this purpose, it is necessary that the at least one first metadata attribute is correct and complete. Furthermore, it is necessary that the at least one metadata attribute is unified. Hence, it is known in advance, that when applying the application to the medical image, it is necessary, that the at least one first metadata attribute comprising a specific attribute tag is correctly filled. The application can pre-define the at least one first metadata attribute which it needs as input value, by the corresponding attribute tag. Hence, the at least one first metadata attribute can be determined based on the corresponding attribute tag. With other words, the at least one first metadata attribute can be chosen respectively selected out of the plurality of metadata attributes based on the attribute tag provided respectively pre-defined by the application.

Preferably, the step applying the first trained function comprises determining or is configured to determine the standardized attribute value based on the other metadata attributes, other attribute tags and/or other provisional or final attribute values. For example, the provisional attribute value of attribute tag “Observation area” and of the first metadata attribute is filled with “Hu%§ &§ fte” originally German “Hüfte”, wherein the first trained function is configured to determine based on the other metadata attributes (e.g., the image acquisition process and setting) as the standardized value “Hips”.

The plurality of metadata attributes can be designed as above described. Each of the metadata attributes of the plurality of metadata attributes comprises an attribute tag and an attribute value. The at least one first metadata attribute and the at least one second metadata attribute are each one of the plurality of metadata attributes.

In a step of receiving at least one second metadata attribute the at least one second metadata attribute is deceived by the interface. The at least one second metadata attribute can be determined and provided by the claimed method in advance. The at least one second metadata attribute is related to the at least one first metadata attribute. In particular, the at least one second and the at least one first metadata attribute are in the same field respectively context. In particular, the at least one second metadata attribute relates to a more generalized aspect of the aspect described by the at least one first metadata attribute. For example, the at least one second metadata attribute comprises the attribute tag ‘body part’. Then the corresponding attribute value can be ‘thorax’. The at least first metadata attribute in this context can comprise the attribute tag ‘organ’. Then the corresponding final attribute value can be ‘heart’. Hence, the at least one second metadata attribute can be used as pre-knowledge for determining the final attribute value. In this example, the first trained function can use the knowledge that the searched organ has to be in the thorax for determining the standardized attribute value. In this case, it is known in advance that the attribute value of the at least one first metadata attribute cannot be ‘brain’ if the attribute value of the at least one second metadata attribute is ‘thorax’. Hence, the first trained function is applied to the medical image and the at least one second metadata attribute.

In another example, the at least one second metadata attribute can comprise ‘organ’ as attribute tag and ‘heart’ as attribute value. Then the first trained function can recognize that the standardized attribute value of the at least one first metadata attribute comprising the attribute tag ‘laterality’ should be empty, as the heart shows no laterality.

The inventors recognized that the knowledge of at least one second metadata attribute which is in the same field as the at least one first metadata attribute can be used as pre-knowledge for determining the final attribute value. In particular, the at least one second metadata attribute value can be used as input for the first trained function. Furthermore, they recognized that the at least one second metadata attribute can be used to determine whether it makes sense to determine the standardized attribute value of the at least one first metadata attribute.

Preferably the step applying a first trained function to the medical image data comprises detecting and/or extracting input data from the medical image data. The input data comprise information associated with the protocol, study description, series description or body region. Herein the first trained function is configured to determine the standardized attribute value based on the input data. The first trained function for example extracts and/or determines the input data based on the other metadata attributes, the attribute tags, the attribute values and/or by analyzing the medical image. The input data can comprise for example information about the modality of image acquisition, the settings of image acquisition, patent information, diagnosis, findings and/or lab results.

Preferably, the step applying a first trained function to the medical image data comprises translating the provisional attribute value into a standardized form and/or standardized language. In other words, the step applying the first trained function and/or the first trained function is configured to translate the provisional attribute value into standardized form, standardized language and/or standardized ontology or dictionary. Particularly, the first trained function is configured to translate the provisional attribute value based on the RadLex dictionary into the standardized attribute value, wherein the standardized attribute value is element of the RadLex dictionary and/or RadLex language.

In particular, the first trained function is based on a natural language processing algorithm and/or comprises a natural language processing algorithm. Preferably, the first trained function is trained to understand grammatical rules, meaning and/or context. In particular, the first trained function is configured to determine the standardized attribute value by analyzing and/or processing the medical image data based on the natural language processing algorithm, grammatical rules, meaning and/or context. The first trained function is in other words configured to analyze the provisional attribute values based on a natural language processing algorithm, especially to analyze the string or text.

Preferably, the medical image data is provided by a local system. The local system is especially part of the unifying system. The local system is for example part of the IT-infrastructure of a hospital. Especially, the local system comprises or is configured as the modality for image acquisition, e.g., the local system comprises a tomography device or is configured as a tomography device. The local system carries out the step checking whether the provisional attribute value is in a standardized form and/or standardized language. Furthermore, the local system is configured to check if the provisional attribute value is empty or not.

The step of applying the first, second or third trained function can be performed and/or be executed by the local system or a central system, e.g., by a cloud. In particular, the local system provides the medical image data to the central system, if the provisional attribute value is not standardized and/or if the local system can not provide the standardized attribute value, e.g., because there are metadata or side information required. The metadata and/or side information are for example saved in the central system, e.g., in a blob storage. The central system, e.g., the cloud, is preferably configured to apply the first trained function to the provided medial image data and to determine the standardized attribute value. The central system is preferably configured to provide the standardized value to the local system, wherein the local system determines the final attribute value based on the provided standardized value and the provisional attribute value. Alternatively, the central system is configured to determine the final attribute value based on the provisional attribute value and the standardized attribute value, wherein the central system provides the final attribute value to the local system.

According to an optional aspect of one or more example embodiments of the present invention, a step of selecting the first trained function from a plurality of first trained functions is based on the at least one second attribute.

In particular, according to the example above, a first trained function can be selected which is suited to identify the imaged organ within a thorax in the medical image. With other words, the at least one second metadata attribute can provide an information about the field respectively the context in which the first trained function should be able to fill in the at least one first metadata attribute. In the example above, the at least one second metadata attribute defines the body region for which the first trained function should be specialized in order to determine the imaged organ for determining the standardized attribute value. With other words, the at least one second metadata attribute can be used as information about the field respectively the context, the first trained function should be specialized in for determining the standardized attribute value of the at least one first metadata attribute.

The inventors recognized that the at least one second metadata attribute can be suited for selecting the correct first trained function out of a plurality of trained functions. With other words, the inventors recognized that the at least one second metadata attribute can define the context respectively the field the at least one first metadata attribute is in. The first trained function should be suited to determine the standardized attribute value with regard to the respective context respectively field.

According to a further aspect of one or more example embodiments of the present invention the provisional attribute value comprises a free-text attribute value. Therein the method is configured for standardizing the free-text attribute value by applying the first trained function to the at least one first metadata attribute, wherein the free-text attribute value is replaced by the standardized attribute value in the provisional and/or final attribute value of the at least one first metadata attribute.

The free-text attribute value can comprise a string value. The free-text attribute value can be provided manually by the medical doctor and/or the medical assistant. The free-text attribute value can comprise at least one not standardized expression and/or at least one abbreviation and/or at least one acronym.

The first trained function can be designed to perform a clinical word sense disambiguation. The first trained function can in particular be designed to replace an attribute value comprising e.g. an acronym and/or an abbreviation by a more standardized attribute value without manipulating the meaning of the attribute value. The first trained function can be based on a natural language processing technique like word embedding, word-level convolutional neural network, bi-direction long short-term memory network, recurrent neural network, dictionary learning and/or an image transformer network. Alternatively or additionally, the first trained function can be based on a machine learning technique like support vector machine, Bayesian model, decision tree and/or k-means clustering. Alternatively or additionally, the first trained function can be based on any other traditional text analyses or comparison technique like an edit-based similarity metric like Levenshtein distance and/or a token-based similarity metric and/or a sequence-based similarity matric and/or a phonetic approach.

The standardized attribute value can be based on a standard ontology like RadLex and/or SNOMED-CT. Alternatively or additionally the standardized attribute value can be determined based on a privately defined dictionary respectively lexicon. The privately defined dictionary can be specific for the medical doctor or for a clinic respectively hospital. The privately defined dictionary can comprise individual abbreviations and/or expressions of the medical doctor and their general respectively standardized pendant. An example for a free-text attribute value can be ‘Thx p.a. LR ER’. The corresponding standardized attribute value can be ‘Thorax Posterior Anterior Left Right Emergency Room’. Another example for a free-text attribute value can be ‘Chest AP Port’. The corresponding standardized attribute value can be ‘Chest anterior posterior portable’.

The provisional attribute value comprising the standardized attribute value can be used for determining the final attribute value as described above. In particular, the at least one second metadata attribute can be determined by standardizing an attribute value of the at least one second metadata attribute as described above. In particular, standardizing the attribute value of the at least one second metadata attribute can be performed in advance, before determining the standardized attribute value of the at least one first metadata attribute.

The inventors recognized that for correcting the provisional attribute value by determining a standardized attribute value and by comparing the standardized attribute value, it might be helpful to first standardize the provisional attribute value. Like this, comparing the attribute values is easier. Furthermore, even if the provisional attribute value is set to be the final attribute value, for further processing the medical image based on the corresponding at least one first metadata attribute, it might be helpful if the final attribute value is standardized. Like this, the final attribute value can be used as input for an application as described above and/or for querying based on the final attribute value.

According to a further aspect of one or more example embodiments of the present invention the method further comprises a step of categorizing the standardized attribute value by applying a second trained function to the at least one metadata attribute. Therein the standardized attribute value is replaced by the categorized standardized attribute value in the provisional attribute value of the at least one first metadata attribute.

In general, the second trained function can be designed as described according to the first trained function. The second trained function can be designed to classify the unstructured text comprised by the standardized attribute value into pre-defined categories like anatomical entity, clinical finding, imaging modality, imaging observation, procedure and/or non-anatomical entities etc. In particular, the single words comprised by the standardized attribute value can be categorized by applying the second trained function to the standardized attribute value. In particular, each word or each expression comprised by the standardized attribute value can be assigned to a category.

The second trained function can be based on clinical named entity recognition. The second trained function can be based on a linguistic grammar technique using a natural language processing technique like word embedding, word-level convolutional neural network, bi-direction long short-term memory network, recurrent neural network and or dictionary learning. Alternatively or additionally, the second trained function can be based on a machine learning technique like support vector machine, Bayesian model, decision tree and/or k-means clustering. Alternatively or additionally, the second trained function can be based on any other traditional text analyses or comparison technique like an edit-based similarity metric like Levenshtein distance and/or a token-based similarity metric and/or a sequence-based similarity matric and/or a phonetic approach.

In the above described example, the expressions of the standardized attribute value ‘Thorax Posterior Anterior Left Right Emergency Room’ can be categorized as following: [Thorax]→‘Anatomy’, [Posterior Anterior]→‘View Position’, [Left Right]→‘Patient Orientation’, [Emergency Room]→‘Location’. The standardized attribute value ‘Chest Anterior Posterior Portable’ of the other example from above can be categorized as following: [Chest]→‘Anatomy’, [Anterior Posterior]→‘View Position’, [Portable]→‘Modality’.

The provisional attribute value comprising the categorized standardized attribute value can be used for determining the final attribute value as described above. In particular, the at least one second metadata attribute can be determined by categorizing and/or standardizing an attribute value of the at least one second metadata attribute as described above. In particular, categorizing and/or standardizing the attribute value of the at least one second metadata attribute can be performed in advance, before determining the standardized attribute value of the at least one first metadata attribute.

The inventors recognized that for further processing the medical image data and/or the at least one first metadata attribute, it might be helpful if the expressions of the standardized attribute values are categorized. In particular, this can help searching respectively querying a plurality of medical images with associated metadata attributes. Furthermore, the categorized standardized attribute value can be used to fill in the attribute values of other metadata attributes associated with the medical image according to the category. With other words, the attribute value of a metadata attribute whose attribute tag is related to a category can be filled in by the standardized expression which is related to the category within the standardized attribute value.

According to a further aspect of one or more example embodiments of the present invention the method comprises a step of performing a semantic matching of the categorized standardized attribute value with higher-level terms by applying a third trained function to the at least one metadata attribute. Therein the categorized standardized attribute value is replaced by the matched categorized standardized attribute value in the provisional attribute value of the at least one first metadata attribute.

In an alternative embodiment a matched standardized attribute value is determined by applying the third trained function to the at least one first metadata attribute, wherein the corresponding provisional attribute value comprises the standardized attribute value. Therein the standardized attribute value is replaced by the matched standardized attribute value in the provisional attribute value of the at least one first metadata attribute.

The third trained function is based on semantic matching. In particular the third trained function is designed to map semantically similar terms to a standardized text e.g. based on a standard ontology like RadLex and/or SNOMED-CT and/or based on a privately defined dictionary. In general, the third trained function can be designed according to the description of the first trained function above. The third trained function can be based on a natural language processing technique like word embedding, word-level convolutional neural network, bi-direction long short-term memory network, recurrent neural network and or dictionary learning. Alternatively or additionally, the third trained function can be based on a machine learning technique like support vector machine, Bayesian model, decision tree and/or k-means clustering. Alternatively or additionally, the third trained function can be based on any other traditional text analyses or comparison technique like an edit-based similarity metric like Levenshtein distance and/or a token-based similarity metric and/or a sequence-based similarity matric and/or a phonetic approach.

The higher-level terms describe the expressions comprised by the categorized standardized attribute value respectively the standardized attribute value in a more general respectively unified way. In the example above the categorized standardized attribute value is ‘Thorax Posterior Anterior Left Right Emergency Room’. The categories have been omitted to enhance the clarity. The matched categorized standardized attribute value is ‘Chest Posterior Anterior Left Right Emergency Room’. Hence, the expression ‘Thorax’ is replaced by the more general higher-level term ‘Chest’. In the other example the categorized standardized attribute value is ‘Chest Anterior Posterior Portable’. Again, the categories are omitted due to clarity. The matched categorized standardized attribute value is ‘Chest Anterior Posterior Portable X-Ray’. Hence, the expression ‘Portable’ is replaced by the higher-level term ‘Portable X-Ray’ as it is defined in an ontology.

The provisional attribute value comprising the matched categorized standardized attribute respectively the matched standardized attribute value can be used for determining the final attribute value as described above. In particular, the at least one second metadata attribute can be determined by matching and/or categorizing and/or standardizing an attribute value of the at least one second metadata attribute as described above. In particular, matching and/or categorizing and/or standardizing the attribute value of the at least one second metadata attribute can be performed in advance, before determining the standardized attribute value of the at least one first metadata attribute.

The inventors recognized that searching respectively querying in a plurality of medical images associated to metadata attributes comprising a matched categorized standardized attribute value or a matched standardized attribute value is more convenient as the single expressions of the attribute values are all comparable.

The steps of standardizing the free-text attribute value, of categorizing the standardized attribute value and of performing a semantic matching of the categorized standardized attribute value can be applied for metadata attributes which are not associated to a medical image data like Health Level 7 (HL7) messages or Fast Healthcare Interoperability Resources (FHIR). In this case, the steps which are based on an analysis of the medical image data can be omitted in order to determine the final attribute value. In particular, in this case, the standardized attribute value or the categorized standardized attribute value or the matched categorized standardized attribute value can be set to be the final attribute value.

In a second aspect one or more example embodiments of the present invention relates to a unifying system for providing at least one first metadata attribute associated with medical image data. The unifying system comprises an interface and a computation unit. Therein the interface is configured for receiving the medical image data and the at least one first metadata attribute. Therein the at least one first metadata attribute comprises an attribute tag and a provisional attribute value. Therein the computation unit is configured for applying a first trained function to the medical image data so as to determine an standardized attribute value. Therein the computation unit is configured for determining a final attribute value based on the provisional attribute value and the standardized attribute value. Therein the interface is configured for providing the at least one metadata attribute. Therein the at least one metadata attribute comprises the attribute tag and the final attribute vale.

In particular, the unifying system can be configured to execute the previously described method for providing at least one first metadata attribute associated with medical image data. The unifying system is configured to execute this method and its aspects by the interface and the computation unit being configured to execute the corresponding method steps. In particular, the interface can comprise one or more sub-interfaces. In particular, the computation unit can comprise one or more computation sub-units.

In a third aspect one or more example embodiments of the present invention relates to a computer program product with a computer program and a computer-readable medium. A mainly software-based implementation has the advantage that even previously used unifying systems can be easily upgraded by a software update in order to work in the manner described. In addition to the computer program, such a computer program product can optionally include additional components such as documentation and/or additional components, as well as hardware components such as e.g. hardware keys (dongles etc.) for using the software.

In a further aspect one or more example embodiments of the present invention relates to a computer program product comprising program elements directly loadable into a memory unit of a first providing system, which induces the unifying system to execute the method according to the claimed method and its aspects when the program elements are executed by the unifying system.

In a fourth aspect one or more example embodiments of the present invention relates to a computer-readable storage medium comprising program elements which are readable and executable by a unifying system, to execute the claimed method and its aspects, when the program elements are executed by the unifying system.

In a further optional aspect one or more example embodiments of the present invention relates to a computer-implemented method for providing a first trained function. The method comprises receiving input training data, wherein the input training data comprises medical training-image data and at least one first metadata training-attribute associated with the medical training-image data. The method comprises receiving output training data, wherein the output training data comprises a final training-attribute value. Therein the output training data is related to the input training data. The method comprises training a first function based on the input training data and the output training data. The method comprises providing the first trained function.

The output training data can be determined based on the input training data by manual annotation. With other words, the final training-attribute value of the at least one first training-attribute can be determined by a medical doctor and/or a medical assistant.

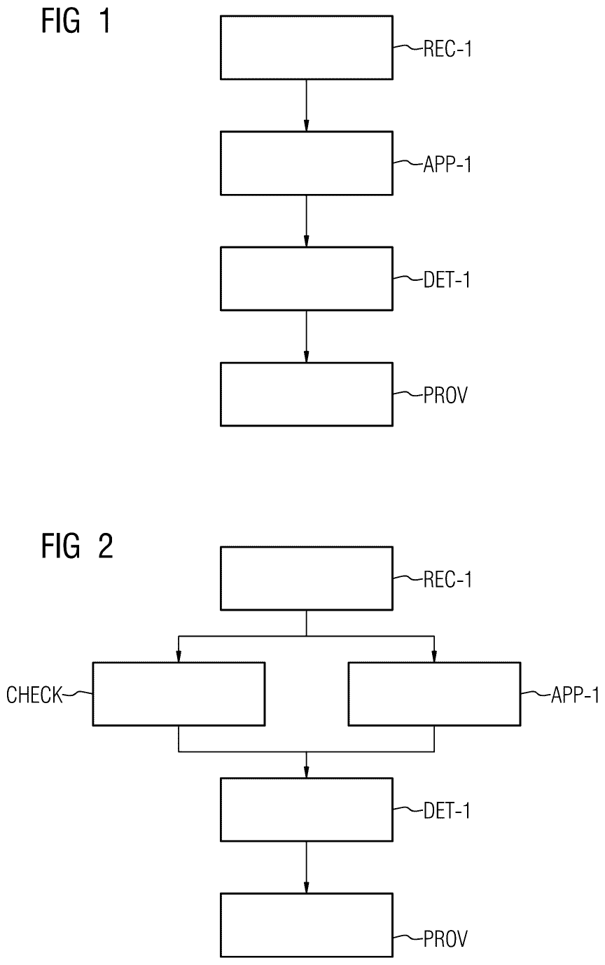

FIG. 1 displays a schematic flow chart of a first embodiment of the method for providing at least one first metadata attribute associated with medical image data.

The medical image is acquired with a medical imaging system. The medical imaging system is configured as a local system and/or part of a local system. The medical imaging system can be for example one out of the following: X-ray system, a Computed-Tomography (CT) system, a Magnetic-Resonance-Imaging (MRI) system, an angiography system, a C-arm system, an ultrasonic system, a Positron-Emission-Tomography (PET) system, a Single-Photon-Emission-Computed-Tomography (SPECT) system. The medical image comprises a pixel matrix or a voxel matrix. Therein the pixel matrix respectively the voxel matrix comprises a plurality of pixels or voxels. Hence, the medical image data can be configured as or can comprise a two-dimensional or a three-dimensional medical image. Alternatively, the medical image can be a four-dimensional medical image. Therein the four-dimensional medical image can in particular comprise a time-series of three-dimensional medical images. The medical image data comprise the medical image.

The medical image depicts an examination object. The examination object is a patient. Alternatively, the examination object can be an animal or an object. The medical image can depict a part of the examination object. For example, the medical image can depict an organ or an extremity of the patient. With other words the medical image can depict a part respectively a body part of the examination object.

The medical image data is associated with at least one first metadata attribute. In this embodiment, the at least one first metadata attribute is a DICOM attribute. The DICOM attribute is comprised by a DICOM header of the medical image. Alternatively, the at least one first metadata attribute can be a NIfTI attribute which is comprised by a NIfTI header. The at least one first metadata attribute characterizes the medical image data. In particular, the at least one first metadata attribute describes which medical imaging system is used for acquiring the medical image, which parameters are used for acquiring the medical image, who or what is imaged, what exactly is the examination object depicted in the medical image, how is the examination object imaged etc. The at least one first metadata attribute comprises an attribute tag and an attribute value. The attribute tag defines the context respectively the field of the at least one metadata attribute. With other words the attribute tag defines what the at least one first metadata attribute refers to. For example, the attribute tag can be ‘Body part examined’, ‘Anatomic Region Sequence’, ‘Patient Orientation’, View Position’, ‘Image Laterality’, ‘Frame Laterality’, ‘Measurement Laterality’ etc. The attribute value can provide the specific value concerning the attribute tag and the medical image. For example, the attribute tag can be ‘Body part examined’. Then the corresponding attribute value can be ‘Thorax’ if the associated medical image comprises an image of a thorax. The at least one first metadata attribute can be for example used to query a database for all medical images which are associated to a metadata attribute comprising the attribute tag ‘Body part examined’ and the attribute value ‘Thorax’. Then these medical images can be compared or further processed. Alternatively or additionally, the at least one first metadata attribute can be used as input value by an application which is applied to the medical data. For example, the application can be configured to provide a diagnosis based on the medical image. In this context, the at least one first metadata attribute can provide an information which body part is shown on the medical image. This information can be used by the application for determining the diagnosis.

In a step of receiving REC-1 the medical image data and the at least one first metadata attribute, the medical image and the at least one first metadata attribute is received by an interface SYS.IF.

The at least one first metadata attribute comprises an attribute tag and a provisional attribute value. The provisional attribute value of the at least one first metadata attribute can be filled automatically during the imaging process or manually by a medical doctor and/or a medical assistant. Alternatively, the provisional attribute value can be empty.

In a step of applying APP-1 a first trained function to the medical image data, the first trained function is applied to the medical image data, especially applied the to metadata attributes, in order to determine a standardized attribute value. The standardized attribute value is related to the attribute tag. The standardized attribute value is, therefore, determined based on the medical image data. For example, the imaged body part can be determined by applying the first trained function to the medical image data, especially the attribute values and attribute tags, if the attribute values of the metadata attributes indicate this. The result of the first trained function is the standardized attribute value.

The first trained function can determine the standardized attribute value based on an ontology or a privately defined dictionary. The ontology can for example comprise RadLex and/or SNOMED-CT. The privately defined dictionary can be defined by a medical doctor and/or a medical assistant and/or for a clinical staff. The privately defined dictionary can comprise terms and expressions typically used by the medical doctor and/or the medical assistant and/or the medical staff.

In a step of determining DET-1 a final attribute value, the final attribute value is determined based on the provisional attribute value and the image-based attribute value. In particular, the final attribute value can be determined to be the image-based attribute value. With other words, the final attribute value can be set to be the image-based attribute value.

In a step of providing PROV the at least one first metadata attribute, the at least one first metadata attribute comprising the attribute tag and the final attribute value is provided via the interface SYS.IF. In particular, the at least one first metadata attribute is provided in association with the medical image data. The at least one first metadata attribute can be used by the application for further processing of the medical image. Alternatively or additionally, the at least one first metadata attribute can be used to compare or query a plurality of medical images within a database, wherein the medical image is one of the plurality of medical images.

FIG. 2 displays a schematic flow chart of a second embodiment of the method for providing at least one first metadata attribute associated with medical image data.

The steps of receiving REC-1 the medical image and the at least one first metadata attribute, of applying APP-1 the first trained function, of determining DET-1 the final attribute value and of providing PROV the at least one first metadata attribute are performed in the same manner as described according to FIG. 1.

The embodiment of the method comprises a step of checking CHECK whether the provisional attribute value is standardized and/or according to RadLex. Furthermore, the step checking CHECK can comprise checking, whether the provisional attribute value is empty or not.

As described above, the provisional attribute value can be filled in automatically or manually during the imaging process. In this case, it should be checked whether the provisional attribute value is correct (standardized) and/or if it fits to the medical image. This can be done by applying the first trained function and/or to compare the provisional attribute value to the standardized attribute value. Hence, in this case the final attribute value can depend on the provisional attribute value and the standardized attribute value. The final attribute value should comprise the correct attribute value.

Alternatively, the provisional attribute can be already standardized. In this case, it is not necessary to apply the first trained function to the medical image data to determine the standardized attribute value. In this case the provisional attribute value can be kept and used as final attribute value. In case the provisional attribute value is empty or not standardized, the step applying APP-1 the first trained function is executed.

Hence, the step of determining DET-1 of the final attribute value depends on the result of the step of checking CHECK whether the provisional attribute value is empty and/or standardized.

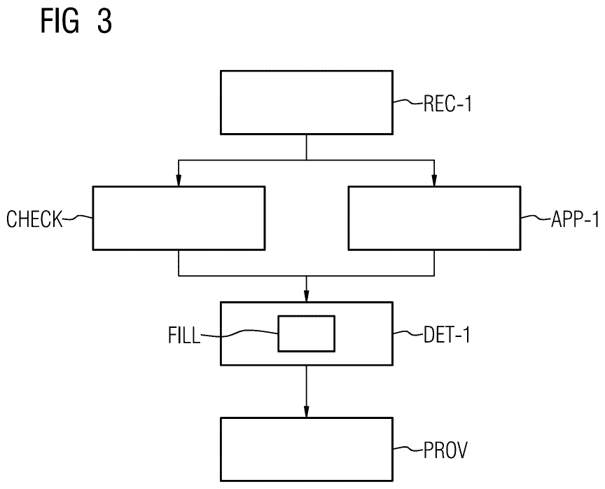

FIG. 3 displays a schematic flow chart of a third embodiment of the method for providing at least one first metadata attribute associated with medical image data.

The steps of receiving REC-1 the medical image data and the at least one first metadata attribute, of applying APP-1 the first trained function, of determining DET-1 the final attribute value and of providing PROV the at least one first metadata attribute are performed in the same manner as described according to FIG. 1. The step of checking CHECK whether the provisional attribute value is empty is performed according to FIG. 2.

In this embodiment, the step of determining DET-1 the final attribute value comprises a step of filling FILL the final attribute value with the standardized attribute value. The step of filling FILL the final attribute value with the standardized attribute value is applied in dependence of the result of the step of checking CHECK whether the provisional attribute value is empty or non-standardized. If this check is positive, the final attribute value filled with the standardized attribute value. With other word, if the provisional attribute value is empty or non-standardized, the final attribute value is set to be the standardized attribute value.

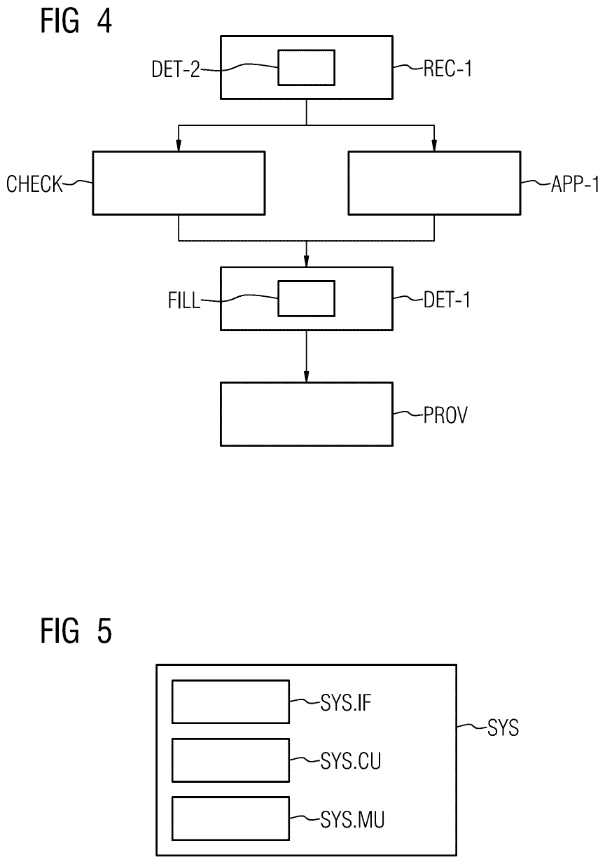

FIG. 4 displays a schematic flow chart of a further embodiment of the method for providing at least one first metadata attribute associated with medical image data.

The steps of receiving REC-1 the medical image data and the at least one first metadata attribute, of applying APP-1 the first trained function, of determining DET-1 the final attribute value and of providing PROV the at least one first metadata attribute are performed in the same manner as described according to FIG. 1. The step of checking CHECK whether the provisional attribute value is empty is performed according to FIG. 2. The step of filling FILL the final attribute value with the standardized attribute value is performed according to FIG. 3.

In this embodiment, the step of receiving REC-1 the medical image and the at least one first metadata attribute comprises a step of determining DET-2 the at least one first metadata attribute out of a plurality of metadata attributes based on an application configured to process the medical image data and/or on the attribute tags.

The plurality of metadata attributes is associated to the medical image data. Each metadata attribute comprises an attribute tag and an attribute value. Therein the attribute tag describes a property of the medical image the corresponding metadata attribute refers to and the corresponding attribute value provides a value according to this property. Alternatively, the attribute value can be empty. The at least one first metadata attribute is one of the plurality of metadata attributes. The plurality of metadata attributes can be received in a separate step.

In the step of determining DET-2 the at least one first metadata attribute, the at least one first metadata attribute is determined out of the plurality of metadata attributes. In particular, the at least one first metadata attribute can be a metadata attribute that is needed for further processing the associated medical image data. For example, the further processing can be performed by an application. The application can be configured to process the medical image. For example, the application can be configured to make some diagnosis and/or some predictions respectively prognosis based on the medical image. The application can be configured such that it needs the at least one first metadata attribute as input data respectively as input value. For example, the application for providing some diagnosis based on the medical image needs an information about the laterality as input data. Thus, the application can for example differ between a broken bone in the right or in the left hand. This information can be provided by the metadata attribute comprising the attribute tag ‘Laterality’ and the corresponding attribute value. Hence, it is essential that the corresponding attribute value is correct and that it is not empty when applying the described application. Hence, the respective metadata attribute can be determined to be the at least one first metadata attribute. Hence, the at least one first metadata attribute can be a metadata attribute which is pre-defined by an application that should be applied afterwards to the medical image and that needs the at least one first metadata attribute as input data.

Alternatively or additionally, the attribute tags of the plurality of metadata attributes can be considered when determining DET-2 the at least one first metadata attribute. In particular, not all metadata attributes might be suitable for being filled and/or corrected by the method described above. For example, an information about the date of acquiring the medical image can be comprised by a metadata attribute of the plurality of metadata attributes. With other words, the corresponding metadata attribute can comprise ‘Acquisition date’ as attribute tag and the exact date as attribute value. It cannot be checked based on the medical image whether the attribute value is correct. Furthermore, the exact date of acquisition cannot be determined based on the medical image by applying the method described above if the attribute value is empty. Hence, this exemplary metadata attribute is not suited to be autofilled and/or corrected by applying the method described above. Hence, based on the attribute tags, only such metadata attributes can be determined out of the plurality of metadata attributes to be potentially the at least one first metadata attribute whose attribute values can be autofilled and/or corrected by applying the method described above based on the medical image. With other words, the at least one first metadata attribute is determined in dependence of the corresponding attribute tag.

FIG. 5 displays a unifying system SYS. The displayed unifying system SYS is configured to execute a method according to one or more example embodiments of the present invention for providing at least one first metadata attribute associated with medical image data. The unifying system SYS comprises an interface SYS.IF, a computation unit SYS.CU, and a memory unit SYS.MU.

The unifying system SYS can in particular be a computer, a microcontroller or an integrated circuit. Alternatively, the unifying system SYS can be a real or a virtual network of computers (a technical term for a real network is “cluster”, a technical term for a virtual network is “cloud”). The unifying system SYS can also be designed as virtual system that is executed on a computer, a real network of computers or a virtual network of computers (a technical term is “virtualization”).

An interface SYS.IF can be a hardware or software interface (for example PCI bus, USB or Firewire). A computation unit SYS.CU can have hardware elements or software elements, for example a microprocessor or a so-called FPGA (acronym for “field programmable gate way”). A memory unit SYS.MU can be implemented as a non-permanent working memory (random access memory, RAM for short) or as a permanent mass storage device (hard disk, USB stick, SD card, solid state disk).

The interface SYS.IF can in particular comprise a plurality of sub-interfaces which carry out different steps of the respective method. In other words, the interface SYS.IF can also be understood as a plurality of interfaces SYS.IF. The computation unit SYS.CU can in particular comprise a plurality of sub-computing units which carry out different steps of the respective method. In other words, the computation unit SYS.CU can also be understood as a plurality of computation units SYS.CU.

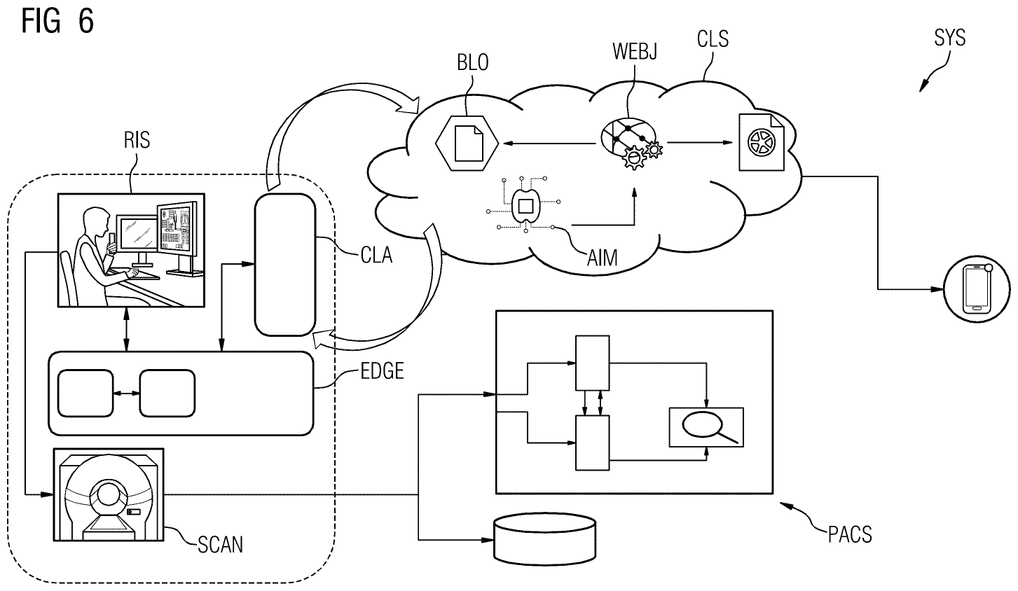

FIG. 6 shows another example of a unifying system SYS. The unifying system SYS comprises six high-level logical components viz. A Scanner SCAN, an EDGE computing system EDG, a Cloud Adapter CLA, a Cloud Service CLS (to identify, decode and map to standard), a Blob Storage BLO and a AI model AIM. Data may be also stored in a PACS-System PACS.

The unifying system SYS enables a cloud-based solution using AI/ML algorithm for finding and translating DICOM tag values in electronic document to the standard ones. During scanning, operator may fill the incorrect values or nonstandard values while creating the secondary capture image. The incorrect secondary image will get burn inside the DICOM file. This may contain nonstandard text values, which is not usable for many Radiologists. This leads to a problem for many institutes and radiologists to analyze the problem and in turn can create difficulty for medical experts in providing solution for many critical or emergency situations in healthcare.

The unifying system SYS takes the medical image data, especially the metadata attributes (e.g., procedure details) as the input and decodes and translates the tag values (protocol, study description, series description, body region) to the standard/required form. Then these translated values are written into the DICOM file (RDSR) and along with the all the header data mentioned. So, the scanned procedure details coming from the RIS will be sent to the EDGE computing system EDG, where the first level analysis, and analytics will be handled. This local computation will give faster result and more secure. If the EDGE system EDG couldn't get the translation done, then the same procedure details is sent to Cloud service CLS with the help of cloud adaptor CLA installed in the RIS system.

The cloud service CLS which contains the decode and translator Webjob WEBJ, will take the input (medical image data) from cloud adaptor CLA. The webjob WEBJ will analyze and decode the incoming procedure details using the specific character set. The decoded data is passed to trained AI/ML NLP module AIM which will translate the required DICOM tag values as per required standard phraseology of particular domain and group. This can be used as plug-in to all the scanners and we can have a decoder and translator before or just it reaches the PACS. This also improves the efficiency of the generated DICOM to make it more readable, relatable and usable by most of Radiologists.

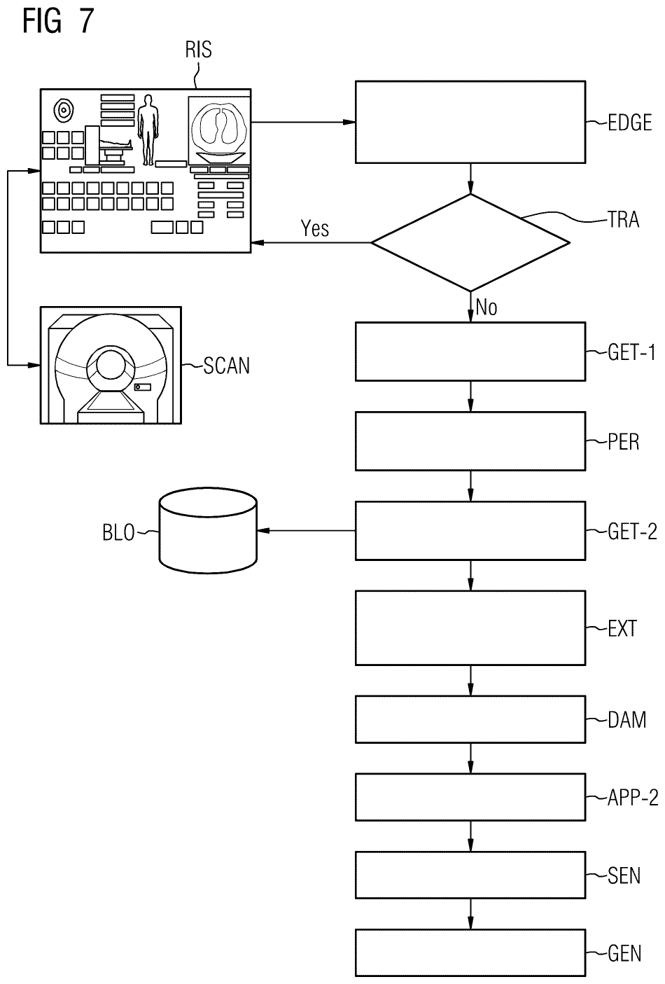

FIG. 7 shows a flow chart of a unifying system SYS applying the method according to one or more example embodiments of the present invention.

An RIS is providing the medical image data, especially input data and/or procedure details to the edge computing system EDG. In Step TRA it is checked if a translation of the provisional attribute value into the standardized attribute value is already available. If it is available the standardized attribute value is provided to the RIS. If it is not available, the step GET-1 is executed. Herein the cloud adapter CLA gets the procedure details, medial image data and/or further metadata attributes and sends it to the cloud services CLS. The procedure details will persist PER in the blob container BLO based on the StudyInstanceUid. In step GET-2 the procedure details are got from the blob storage BLO and are arranged according to instance*. In step EXT all the relevant procedure details and/or further details are extracted and send to decode and translator webjob. After data massage DAM the AIM (e.g., the first trained function) is applied APP-2 to the massaged data, wherein the standardized attribute value is determined. In the step send SEN the standardized attribute value is send to the RIS. In an optional step GEN an electronic document is generated.

EDGE Computing System EDGE is coming up with an ideology of bringing compute, storage and networking closer to the consumer. Edge Computing is to minimize the latency by bringing the public cloud capabilities to the edge.

The edge computing system EDGE takes the medical image data, especially the further metadata attributes and doses a data massage on top of them. The further metadata attributes may comprise information about protocol, study description, series description, requested procedure. The data is analyzed and converted to the appropriate format. Data caching is used to minimize the latency of the response. If the translation fails in the edge computing system EDGE, then only the required data is uploaded to the cloud and makes use of central cloud system CLS to get the translated strings.

The first trained function and/or the AIM is configured for translating the provisional value tag. The first trained function is for example configured to understand different languages and/or to understand the grammatical rules, meaning and context, but also colloquialisms, slang and acronyms used in a language. The first trained function comprises for example a natural language processing algorithm. The first trained function, the AIM or NLP is particularly configured to translate the attribute values from different languages to a common standard.

The method for providing the final attribute value can for example be implemented by the steps:
Fetching procedure details from the blob storage.
Analyzing whether the procedure details need the translation or in appropriate format.
Examining if the translated strings are already available in the look up table or map.
If string is available, then sending the translated string back to the adaptor.
If strings not available, then decoding the data based on specific character set.
Web job then sends the required information to the trained AI/ML module AIM for translation.
AIM uses neural machine translation model and neural algorithms the detect the relationship between data set, it mimics the process of hoe human brain operates to detect the similar text of any other language in same context.
Translated strings will be send back to RIS via cloud adaptor CLA.
Also translated strings will be stored in the database for forthcoming analysis of procedure details.
Translator is configurable through configuration file.
WebJob sends a notification to the radiologist about the translated data.

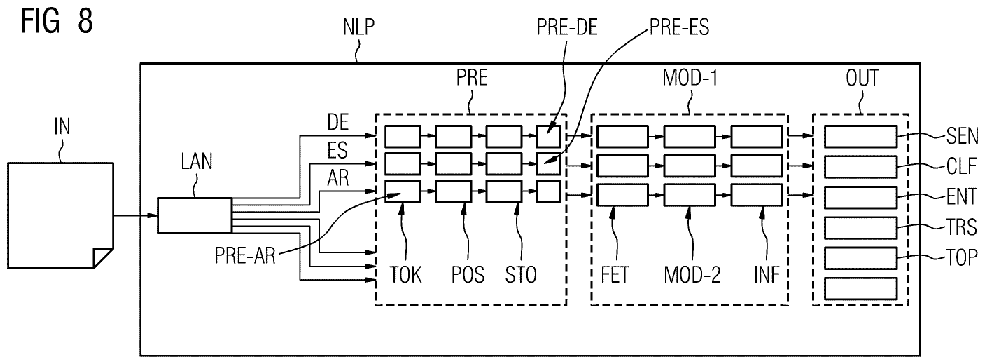

FIG. 8 shows an example of an Natural language processing algorithm NLP, wherein the Natural language processing algorithm NLP can be comprised by the first trained function. The Natural language processing algorithm NLP is provided with input data IN. The input data IN are based on the metadata attributes of the medical image data. The input data IN comprise for example information and/or attribute values about the protocol, study description, body region, series description and/or requested procedure. In a language deduction step LAN an language of the input data IN is determined. The language detection LAN is preferably carried out based on deep learning and/or machine learning. After determining the language, e.g., German DE, Spanish ES or Arabic AR a pre-processing step PRE is carried out.

Particularly, based on the determined language different pre-processing steps PRE or versions of the pre-processing step PRE are carried out. E.g., a German pre-processing step PRE-DE, a Spanish pre-processing step PRE-ES or Arabic pre-processing step PRE-AR can be used. In the pre-processing step PRE a tokenization TOK, PoS Tagging POS and/or stop word removal STO is applied on the input data IN.

Based on the pre-processing step PRE and/or its output data a modelling step MOD is carried out. The modelling step can also be different for the language, e.g. a German modelling step MOD-DE, a Spanish modelling step MOD-ES and/or a Arabic modelling step MOD-AR. The modelling step MOD comprise for example a feature extraction FET, Modelling MOD-2 and/or Inference INF. Based on the modelling step MOD-1 output data OUT are generated. The output data OUT comprise for example a sentiment SEN, a classification CLF, an entity extraction ENT, translation TRS and/or topic modelling TOP. Based on the output data OUT the standardized value is determined.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

Wherever not already described explicitly, individual embodiments, or their individual aspects and features, can be combined or exchanged with one another without limiting or widening the scope of the described invention, whenever such a combination or exchange is meaningful and in the sense of this invention. Advantages which are described with respect to one embodiment of the present invention are, wherever applicable, also advantageous of other embodiments of the present invention.

1. A computer-implemented method for providing at least one first metadata attribute of medical image data, the method comprising:
receiving the medical image data and the at least one first metadata attribute, the at least one first metadata attribute including an attribute tag and a provisional attribute value;
applying a first trained function to the medical image data to determine a standardized attribute value;
determining a final attribute value based on the provisional attribute value and the standardized attribute value; and
providing the at least one first metadata attribute, the at least one first metadata attribute including the attribute tag and the final attribute value.

2. The method of claim 1, further comprising:
checking whether the provisional attribute value is in at least one of a standardized form or a standardized language, the determining determines the final attribute value based on the checking.

3. The method of claim 2, wherein if the provisional attribute value is not in the at least one of the standardized form or the standardized language the determining includes,
filling the final attribute value with the standardized attribute value.

4. The method of claim 1, wherein at least one of the attribute tag or the provisional attribute value is associated with a protocol, a study description, a series description or a body region.

5. The method of claim 4, wherein the applying includes,
at least one of detecting or extracting input data from the medical image data, wherein the input data comprise information associated with the protocol, the study description, the series description or the body region, and the first trained function is configured to determine the standardized attribute value based on the input data.

6. The method of claim 1, wherein the applying includes,
translating the provisional attribute value into at least one of a standardized form or a standardized language.

7. The method of claim 1, wherein
the first trained function comprises at least one of a natural language processing algorithm or is trained to understand at least one of grammatical rules, a meaning or a context, and
the determining the standardized attribute value includes analyzing or processing, by the first trained function, the medical image data based on the at least one of the natural language processing algorithm, the grammatical rules, the meaning or the context.

8. The method of claim 2, wherein the medical image data are provided by a local system, and the checking is performed by the local system.

9. The method of claim 1, wherein the medical image data is provided by a local system, the local system provides the medical image data to a cloud system, the applying is performed by the cloud system.

10. A unifying system for providing at least one metadata attribute associated with medical image data, the system comprising:
an interface configured to receive the medical image data and the at least one metadata attribute, the at least one metadata attribute including an attribute tag and a provisional attribute value; and
a computation unit configured to,
apply a first trained function to the medical image data to determine a standardized attribute value,
determine a final attribute value based on the provisional attribute value and the standardized attribute value,
wherein the interface is configured to provide the at least one metadata attribute, the at least one metadata attribute including the attribute tag and the final attribute value.

11. A non-transitory computer program product comprising program elements, when executed by a unifying system, cause the unifying system to perform the method of claim 1.

12. A non-transitory computer-readable storage medium comprising program elements, when executed by a unifying system, cause the unifying system to perform the method of claim 1.

13. The method of claim 3, wherein at least one of the attribute tag or the provisional attribute value is associated with a protocol, a study description, a series description or a body region.

14. The method of claim 13, wherein the applying includes,
at least one of detecting or extracting input data from the medical image data, wherein the input data comprise information associated with the protocol, the study description, the series description or the body region, and the first trained function is configured to determine the standardized attribute value based on the input data.

15. The method of claim 13, wherein the applying includes,
translating the provisional attribute value into at least one of a standardized form or a standardized language.

16. The method of claim 13, wherein
the first trained function comprises at least one of a natural language processing algorithm or is trained to understand at least one of grammatical rules, a meaning or a context, and
the determining the standardized attribute value includes analyzing or processing, by the first trained function, the medical image data based on the at least one of the natural language processing algorithm, the grammatical rules, the meaning or the context.

17. The method of claim 16, wherein the medical image data are provided by a local system, and the checking is performed by the local system.

18. The method of claim 17, wherein the medical image data is provided by a local system, the local system provides the medical image data to a cloud system, the applying is performed by the cloud system.
